(19) United States
(12) Patent Application Publication
(10) Pub. No.: US 2017/0014609 A1
(43) Pub. Date: Jan. 19, 2017

(54) TATTOOING DEVICE

(71) Applicant: Harold Raymond SPADONI III, New Orleans, LA (US)

(72) Inventor: Harold Raymond SPADONI III, New Orleans, LA (US)

(21) Appl. No.: 14/797,873

(22) Filed: Jul. 13, 2015

(51) Int. Cl. A61M 37/00

(52) U.S. Cl. CPC A61M 37/0084

(57) ABSTRACT

An ergonomic tattooing device adapted to be continuously fed with unexposed ink so as not to need refill dipping is provided. The tattooing device may include a main body forming a centrally disposed, elongated cavity fluidly communicating with an attached needle. The main body may form a cartridge cavity fluidly connecting an outer portion of the main body and the elongated cavity, wherein the cartridge cavity is dimensioned to house an ink cartridge adapted to release a droplet of the coloring ink into the elongated cavity at a predetermined sufficient rate.

### BACKGROUND OF THE INVENTION

The present invention relates to tattooing and, more particularly, to an ergonomic tattooing device adapted to be continuously fed with unexposed ink so as not to need refill dipping.

Ever since electric tattooing machines were introduced there have always been questions as to whether the ink is sufficiently sterile, and why tattoo artists have to stop to refill dip for ink so many times. The problem is that ink is prone to capturing small airborne particles that can trigger an allergic reaction in some people. As such particles can fly into an exposed ink setup, tattoo artists need to routinely refill dip for ink using unexposed setups during a single application of a tattoo. Such repeated refill dipping back and forth fatigues the tattoo artist—maybe over 300 times when performing body art on a client for over three hours—straining their neck, shoulder, arm and back; not to mention breaking their concentration. When performing body art, focus is paramount. And so when the artist has to stop and go due to refill dipping he or she may find it hard to focus on some very fine intricate art. This refill dipping also causes stress to the client as they have to adjust to the repeated re-application of the tattoo needle on the skin.

As can be seen, there is a need for an ergonomic tattooing device adapted to be continuously fed with unexposed ink so as not to need refill dipping.

### SUMMARY OF THE INVENTION

In one aspect of the present invention, a tattooing device includes a main body having an operable end; an elongated cavity formed by the main body along its longitudinal axis; an operable end fluidly communicating the operable end and the elongated cavity; an inlet opening disposed along an upper portion of the main body; and a cartridge cavity formed by the main body so as to interconnect the inlet opening to the elongated cavity.

These and other features, aspects and advantages of the present invention will become better understood with reference to the following drawings, description and claims.

### BRIEF DESCRIPTION OF THE DRAWINGS

FIG. 1 is a side elevation view of an exemplary embodiment of component 1 of the present invention;

FIG. 2 is a front elevation view of an exemplary embodiment of component 1 of the present invention;

FIG. 3 is an exploded perspective view of an exemplary embodiment of the present invention;

FIG. 4 is a section view of an exemplary embodiment of the present invention; and

FIG. 5 is a top plan view of an exemplary embodiment of component 1 of the present invention.

### DETAILED DESCRIPTION OF THE INVENTION

The following detailed description is of the best currently contemplated modes of carrying out exemplary embodiments of the invention. The description is not to be taken in a limiting sense, but is made merely for the purpose of illustrating the general principles of the invention, since the scope of the invention is best defined by the appended claims.

Broadly, an embodiment of the present invention provides an ergonomic tattooing device adapted to be continuously fed with unexposed ink so as not to need refill dipping. The tattooing device may include a main body forming a centrally disposed, elongated cavity fluidly communicating with an attached needle. The main body may form a cartridge cavity fluidly connecting an outer portion of the main body and the elongated cavity, wherein the cartridge cavity is dimensioned to house an ink cartridge adapted to release a droplet of the coloring ink into the elongated cavity at a predetermined sufficient rate.

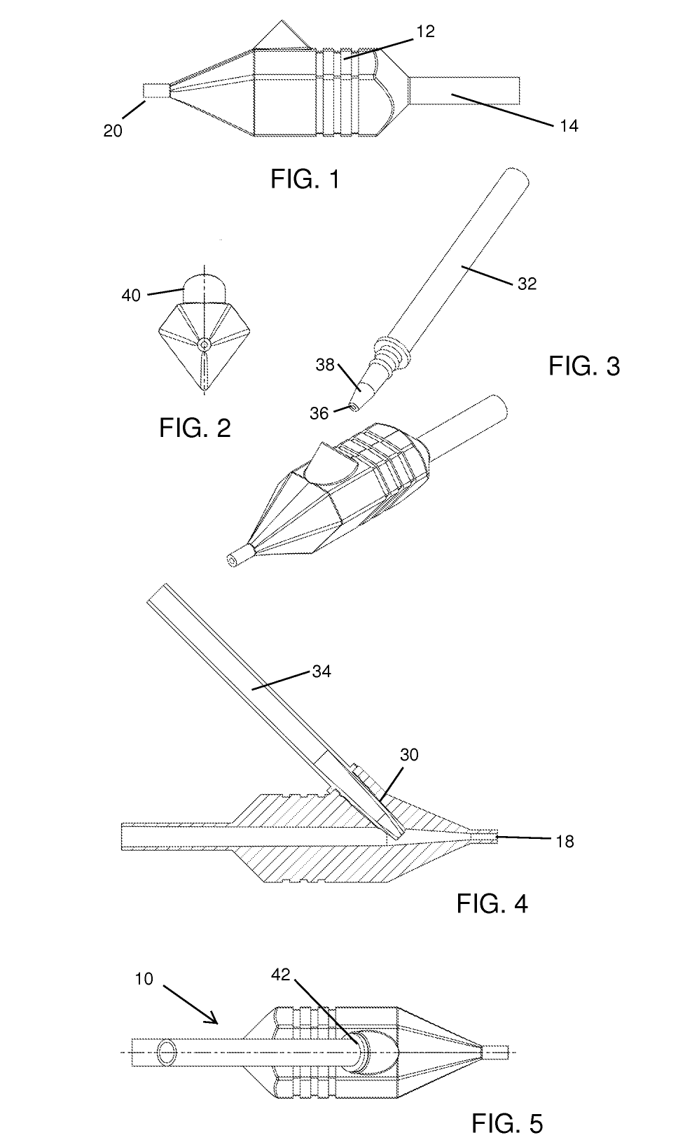

Referring to FIGS. 1 through 5, the present invention may include a tattooing device 10 having a main body 12 and an ink cartridge 32 removably attached thereto. The main body 12 and the ink cartridge 32 may be operably engaged by a separate tattooing machine so that a tattoo artist may ergonomically deposit a coloring ink, via a tattoo needle, into the skin of a customer. The main body 12 may be adapted to be maneuvered manually and so act as a guide for the tattoo needle. The main body 12 may include a second body portion 14. The main body 12 may have an elongated diamond shape as illustrated in the FIGS; however, the main body 12 may have any geometric or non-geometric shape so long as the main body 12 functions in accordance with the present invention as described herein.

The main body 12 may form an elongated cavity 16 centrally disposed along its length and the length of the second body portion 14. The elongated cavity 16 may form an operable opening 18 disposed at an operable end 20 of the main body 12 and a second opening at a terminus of the second body portion. The elongated cavity 16 may taper as it extends to the operable end 20. The terminus of the second body portion may be removably connected to the separate tattoo machine (not shown) by a vice grip, or the like, so that the separate tattoo machine can impart vibratory motion to the tattooing device 10. The operable end 20 may be adapted to removably secure a tattoo needle (not shown) that vibrates via said imparted vibratory motion.

The main body 12 may form a cartridge cavity 30 extending from an inlet opening 42 disposed along an outer portion of the main body 12 to the elongated cavity 16 so as to be in fluid communication thereto. The cartridge cavity 30 may be disposed at an angle relative to the longitudinal axis of the elongated cavity 16. The cartridge cavity 30 may be dimensioned and adapted to operably receive a portion of the ink cartridge 32 therein. A cartridge seat 40 may be disposed along at least a portion of the inlet opening 42 for properly seating the ink cartridge 32.

The ink cartridge 32 may be form a hermetically-sealed or otherwise hypoallergenic ink cavity 34 for releasing a coloring ink from a release opening 36 of its sterile nozzle 38. The coloring ink may include any coloring liquid, pigments, dyes, cosmetic material and the like adapted for tattooing biological tissue. The sterile nozzle 38 and the ink cavity 34 may taper as they extend to the release opening 36. The sterile nozzle 38/release opening 36 may be aligned and operably disposed within the cartridge cavity 30 by the disposition of the cartridge seat 40.

The ink cartridge 32 may be adapted to release a droplet of the coloring ink through its release opening 36 at a sufficient rate, due to at least gravity and the vibrating motion imparted by the separate tattooing machine. As a result, the ink cartridge 32 may be upwardly oriented relative to the main body 12, as illustrated in FIGS. 3 through 5. The sufficient rate of coloring ink droplet release may be predetermined by the separate tattooing machine's adaptable vibratory motion.

A method of using the present invention may include the following. The tattooing device 10 disclosed above may be provided. A user may operably house a portion of the ink cartridge 32 in the cartridge cavity 30. Then the user may operably secure the needle to the operable end 20 of the main body 12. With the separate tattooing machine imparting a predetermined vibratory motion through the tattooing device 10, the ink cartridge 32 may release a droplet of the coloring ink at a sufficient rate so that the coloring ink flows into the elongated cavity 16 and through the operable opening 18, whereby the tattoo artist may ergonomically deposit the coloring ink, via the needle, into the skin of a customer so as not to need refill dipping.

The tattooing device 10 may be stored and sold in sterile packaging. The ink cartridge 32 and needle are disposable subsequent use so as to promote hypoallergenic utilization.

In an alternative embodiment, the present invention may be linked to a computerized printer with the capabilities of producing programmable artwork for leather goods including, but not limited to, sneakers, boots, heels, sandals, belts, handbags, wallets, trunks, luggage, patches, interior design, and the like.

It should be understood, of course, that the foregoing relates to exemplary embodiments of the invention and that modifications may be made without departing from the spirit and scope of the invention as set forth in the following claims.

What is claimed is:

1. A tattooing device, comprising:
a main body having an operable end;
an elongated cavity formed by the main body along its longitudinal axis;
an operable end fluidly communicating the operable end and the elongated cavity;
an inlet opening disposed along an upper portion of the main body; and
a cartridge cavity formed by the main body so as to interconnect the inlet opening to the elongated cavity.

2. The tattooing device of claim 1, further comprising an ink cartridge dimensioned to be removably secured in the cartridge cavity.

3. The tattooing device of claim 2, wherein the ink cartridge is configured to release a droplet of coloring ink based in part on gravity.

4. The tattooing device of claim 3, wherein the ink cartridge is configured to release the droplet of coloring ink based in part on a vibratory motion.

5. The tattooing device of claim 4, further comprising a cartridge seat disposed along a periphery of the inlet opening.

6. The tattooing device of claim 5, wherein the main body is an elongated diamond and cylindrical shape.

7. The tattooing device of claim 6, wherein the cartridge cavity is disposed in an upwardly orientation relative to the elongated cavity.

8. The tattooing device of claim 7, wherein the operable end is configured to secure a tattoo needle.

9. The tattooing device of claim 8, wherein the elongated cavity tapers as it extends to the operable end.

10. The tattooing device of claim 9, wherein the ink cartridge tapers as it extends into the cartridge cavity.
